(19) United States
(12) Patent Application Publication
(10) Pub. No.: US 2019/0074084 A1
(43) Pub. Date: Mar. 7, 2019

(54) CONTEXTUAL FILTERING OF LAB VALUES

(71) Applicant: KONINKLIJKE PHILIPS N.V., EINDHOVEN (NL)

(72) Inventors: Merlijn SEVENSTER, HAARLEM (NL); Paul Joseph CHANG, CHICAGO, IL (US)

(73) Assignee: Koninklijke Philips N.V., Eindhoven (NL)

(21) Appl. No.: 16/084,696

(22) PCT Filed: Mar. 28, 2017
(86) PCT No.: PCT/EP2017/057234

§ 371 Date: Sep. 13, 2018

Related U.S. Application Data
(60) Provisional application No. 62/313,832, filed on Mar. 28, 2016.

(51) Int. Cl. G16H 30/40; G16H 10/40; G16H 15/00; G16H 10/60

(52) U.S. Cl. CPC G16H 30/40; G16H 10/60; G16H 15/00; G16H 10/40

(57) ABSTRACT

A system (100) includes a relevancy computation engine (150) configured to compute a relevancy score for a lab value in a lab report of a patient by applying rules that map one or more patient state indications and the lab value to the relevancy score.

### FIELD OF THE INVENTION

The following generally relates to medical imaging and medical informatics with specific application to interpretation of medical images in light of patient medical laboratory reports.

### BACKGROUND OF THE INVENTION

Healthcare professionals, such as radiologists, review and interpret or read medical images of patients generated by medical imaging scanners. The healthcare professionals are under time pressures to quickly (in minutes) and accurately interpret medical images.

Best practice in reviewing a medical image of a patient is to include review and synthesis of the patient's medical history. This can include imaging orders, prior images, and various medical reports, such as lab reports. A patient can have many lab reports, i.e. a history of reports corresponding to different events. Furthermore, lab reports tend to be voluminous with sparse relevant information. For example, a lab report includes many tests and/or measured values. One conventional approach is to chronologically review reports, e.g. newest to oldest, and sequentially review values in each lab report. The conventional approach is time consuming and can be mentally fatiguing, which contributes to a lack of lab report review by many healthcare professionals.

Conventional approaches to improving the medical imaging review process are typically directed toward toolsets that facilitate review of individual images, e.g. tools that operate directly on the images and/or facilitate access and/or manipulation of the actual images.

### SUMMARY OF THE INVENTION

Aspects described herein address the above-referenced problems and others.

The following describes a method and system for contextual filtering of patient medical lab values from lab reports. Context includes at least one indication of a patient state, which is obtained by semantic analysis of a reason for an examination, such as a reason for a medical imaging study, and/or a semantic analysis of problems in a patient problem list. A relevancy score is computed for a lab value of the patient determined by an evaluation of rules that map the at least one patient state indication and the lab value to a relevancy score. The relevancy scores can be used to filter the lab values.

In one aspect, a system includes a relevancy computation engine configured to compute a relevancy score for a lab value in a lab report of a patient by applying rules that map one or more patient state indications and the lab value to the relevancy score.

In another aspect, a method includes computing a relevancy score for a lab value of a patient by applying rules that map one or more patient state indications and the lab value to the relevancy score.

In another aspect, a system includes a non-transitory storage media containing instructions that when executed by one or more processors are configured to identify and normalize one or more patient state indications of a patient using at least one of a reason for a medical examination and one or more patient medical problems. The non-transitory storage media containing instructions that when executed by one or more processors are further configured to compute relevancy scores for lab values of the patient by applying rules to the identified and normalized one or more patient state indications, wherein the rules map patient state medical indications and medical lab values to relevancy scores. The non-transitory storage media containing instructions that when executed by one or more processors are further configured to display the lab values on a display device filtered by the relevancy score according to a predetermined threshold.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of contextual lab values filtering system.

FIG. 2 flowcharts an embodiment of a method of contextually filtering lab values.

### DETAILED DESCRIPTION OF EMBODIMENTS

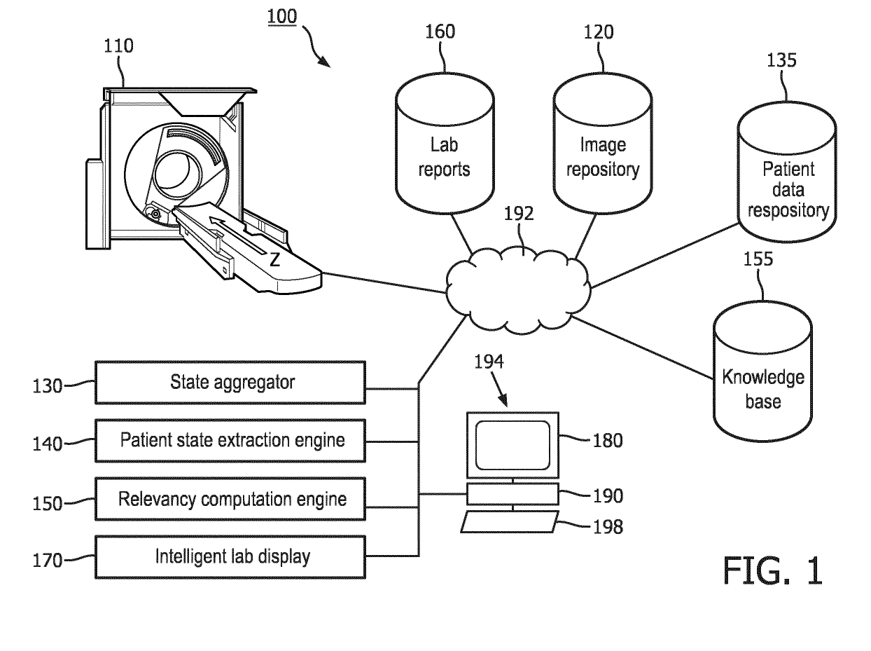

Initially referring to FIG. 1, a contextual lab values filtering system 100 is schematically illustrated. A medical imaging device 110, such as a computed tomography (CT) scanner, a magnetic resonance (MR) scanner, a positron emission tomography (PET) scanner, a single proton emission computed tomography (SPECT) scanner, an ultrasound (US) scanner, combinations and the like, generates a medical image of a patient. The medical image can be stored in an image repository 120, such as a Picture Archiving and Communication System (PACS), Radiology Information System (RIS), Electronic Medical Record (EMR), and the like.

A state aggregator 130 accumulates and manages medical information by patient in a patient data repository 135. The medical information can be accumulated through Health Level Seven (HL7) messages and/or queries of other patient data repositories, such as an EMR, a RIS, a PACS, a lab system, and the like. In some embodiments, the patient data repository 135 can include one or more of the EMR, RIS, PACS, the lab system, and/or portions thereof.

A patient state extraction engine 140 extracts medical information from the patient data repository 135 via the state aggregator 130 for a patient, and identifies and normalizes indications that characterize a patient's medical and/or disease state. For example, a reason for a medical imaging examination can be extracted from an order entry (OE) system, RIS, or PACS system and a patient problem list extracted from the EMR. The reason for the examination and the patient problem list can contain potential patient state indications. In some instances, the reason for the examination includes patient state information that is current or timely characterizes the patient's medical and/or disease state. In some embodiments, the patient state indications can include an anatomy, a modality, a protocol, or other information according to the type of examination. In some instances, information about the examination can provide information that potentially characterizes the patient's medical and/or disease state. In some instances, the patient problems list includes information of a broader view that characterize the patient's medical and/or disease state.

The extracted medical information can include structured data or unstructured data. Structured data can include identification of an ontological concept. For example, structured reports can include ontological concepts according to International Classification of Disease (ICD), RadLex, Systematized Nomenclature of Medicine (SNOMED) codes, which identify the information according to one or more of the ontologies. Other sources of medical information are contemplated based on the healthcare systems configuration.

The extraction from the state aggregator 130 can be initiated by access of the patient medical image, receipt of the patient medical image from the medical imaging device 110 by the image repository 120, a scheduling of a review of the patient medical image by a healthcare professional, an access of the patient records through a subsystem, and the like.

A semantic analysis by the patient state extraction engine 140 identifies ontological concepts in unstructured reports, e.g. narrative text using techniques or tools known in the art. Examples of examples of text analysis and concept extraction, such as “cTakes™” developed by Children's Hospital Boston, or “Metamap” maintained by the U.S. National Library of Medicine. For example, if a reason for the examination is “r/o pneumonia. Cough/fever” (rule out pneumonia, patient exhibits cough and fever), then normalized identified ontological concepts using an ICD-9 (ICD version 9) ontology include viral pneumonia (480), cough (786.2) and fever (780.60). The identified and normalized ontological concepts provide indications of the patient state.

Furthermore, the patient state extraction engine 140 can semantically integrate between different ontologies and/or ontology versions. For example, a semantic analysis of the reason for examination uses the SNOMED ontology and a semantic analysis of problems in the patient problem list uses ICD, and the SNOMED ontological concepts are then mapped to ICD to return a list of acute indications according to a single expected ontology. The mappings for example between ICD-9, ICD-10, SNOMED and/or RadLex can be bidirectional or unidirectional. For example, SNOMED fever (386661006) can be mapped bidirectionally to ICD-9 fever (780.60).

A relevancy computation engine 150 receives the patient state indications and applies rules from a knowledge base 155 that map the indications and lab values in one or more lab reports 160 to relevancy scores. Each lab value can be assigned a relevancy score. The patient state indications and the labs values can include dates and/or ages. For example, the patient state indications can include a date reported, a date entered, a date of the problem experienced by the patient, and the like.

The relevancy computation engine 150 can use hierarchical reasoning to generalize patient state indication using ontological concepts. The hierarchical reasoning uses a “is-a” semantic relationship to generalize the concept within the ontology. For example, a fever (780.60) is a fever and other physiological disturbances of temperature regulation (780.6), which is a general symptom (780), which is a symptom (780-789) within the ICD-9 ontology. A rule based approach can identify those concepts which are patient state indications, such as a symptom of ICD-9 ontology. For example, a fever (780.60), a post procedural fever (780.62), a post vaccination fever (780.63), and chills (without fever) (780.64) can be represented hierarchically as fever (780.6). The relevancy computation engine 150 can implement the hierarchical reasoning using rules, which map each of a fever (780.60), a post procedural fever (780.62), a post vaccination fever (780.63), and chills (without fever) (780.64) to fever (780.6), and using the higher hierarchical level of fever (780.6) with a lab value to determine the relevancy score. A lower hierarchical level can be used.

The relevancy score can be represented in a continuous range, such as a closed interval of [0-1], where 0 is not relevant and 1 is relevant. The relevancy computation engine 150 can reconcile multiple computed scores for the same lab value as a function of a set of scores (the set includes the multiple computed scores), e.g. a maximum, an average, and the like. In one embodiment the relevancy computation engine 150 can assign a relevancy score to a lab value, which is not present in the lab reports, e.g. unknown and relevant. For example, a white blood cell (WBC) count lab value may be relevant, but is not present in any lab report for the patient.

The knowledge base 155 includes rules that map known patient state indications and relevant medical lab values to relevancy scores. The knowledge base 155 can include a non-transitory storage media storing rules, e.g. cloud storage, disk storage, etc. The rules can be constructed manually based on relevant medical lab values corresponding to known patient state indications reported in medical literature. The rules can include time considerations that assign and/or compute a relevancy score as a function of the age of lab values and/or age of the patient state indications. The rules can include mappings of a lab value relative to or as a function of a normal lab value range and/or a non-normal value range.

An example rule can include that if patient state indications include “fever”, then a WBC value in a lab report is relevant, e.g. a relevancy score for “fever” and WBC is 1. Another example rule can include that if patient state indications include “fever” and the “fever” is from an EMR condition in the patient problem list entered 2 years ago, then “fever” can be suppressed in the reasoning, e.g. a relevancy score for WBC and fever with age greater than or equal 2 years is 0. Another example rule can include that if patient state indications include “fever” that is no more than 14 days old, then a WBC value in a lab report is relevant, e.g. a relevancy score for WBC and fever with age less than or equal 14 days is 1. Another example rule can include that if a WBC value is out of normal range, then the WBC value in a lab report is relevant. Rules can be combined, such as if patient state indications include “fever” that is no more than 14 days old and a WBC value is out of normal range, then WBC value is relevant, e.g. rules can include Boolean logic.

A lab display 170 displays lab values on a display device 180 according to the relevancy score. The display can include only relevant lab values, e.g. values with a relevancy score greater than the predetermined threshold. In some instances, displaying only the relevant lab values reduces the number of lab values to be reviewed by the healthcare professional, e.g. fewer than all lab values in a report or reports are displayed, which can improve efficiency of review.

The lab values can be ordered or ranked based on the relevancy scores. For example, the highest ranked lab values according to the relevancy score are displayed first. The display can include the lab values highlighted according to the relevancy score in a displayed lab report. For example, the lab values formatted in a display with color and/or intensity according to the relevancy score. For example, the lab values with a highest relevancy score range can be highlighted in a first color, such as red, in a second range highlighted in a second color, such as yellow, and in a third range highlighted in a third color, such as green, and so forth.

The lab display 170 can use the relevancy score to filter the lab values according to the relevancy score and a predetermined threshold, which are formatted according to another display format. For example, a list of lab values and corresponding relevancy scores greater than the threshold can be returned to a calling program. In another embodiment, the system 100 can receive a patient identification, return patient state indications and/or receive patient state indications and return lab values filtered according to relevancy.

The predetermined threshold can be configurable and personalizable. For example, a predetermined threshold can be based on one or more of the patient state indications, the type of review or examination, policies of a healthcare organization and/or a reviewing healthcare professional, and the like.

The contextual lab values filtering system 100 can operate through an application programming interface (API) associated with a PACS, EMR, RIS or other system. The system can receive a patient identification and return lab values according to the determined relevancy score. The returned lab scores can include a lab display formatted and/or filtered according to the relevancy score.

The state aggregator 130, the patient state extraction engine 140, the relevancy computation engine 150, and the lab display 170 comprise one or more configured processors 190, e.g., a microprocessor, a central processing unit, a digital processor, and the like. The one or more configured processors 190 are configured to execute at least one computer readable instruction stored in a computer readable storage medium, which excludes transitory medium and includes physical memory and/or other non-transitory medium to perform the techniques described herein. The one or more processors 190 may also execute one or more computer readable instructions carried by a carrier wave, a signal or other transitory medium. The one or more processors 190 can include local memory and/or distributed memory. The one or more processors 190 can include hardware/software for wired and/or wireless communications over a network 192. For example, the lines in FIG. 1 indicate communications paths between the various components, which can be wired or wireless. The one or more processors 190 can comprise the computing device 194, such as a desktop, a laptop, a body worn device, a smartphone, a tablet, and/or cooperative/distributed computing devices including one or more configured servers (not shown). The computing device 194 can include the display device 180, which can display the filtered lab values. The computing device 194 can include one or more input devices 198 which receive commands, such as identifying the patient, and/or patient image, displaying the patient state indications, operating aspects of the display of lab values, overlay and/or co-display of patient medical image, etc.

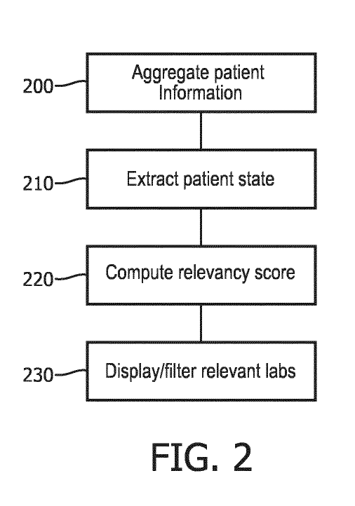

With reference to FIG. 2, an embodiment of a method of contextually filtering lab values is flowcharted. At 200, medical information including one or more patient states are aggregated by the state aggregator 130. The aggregation can occur dynamically, e.g. as the patient is identified for contextually filtering lab values. The aggregation can occur in parallel with other patients and/or with various data sources as they become available to the state aggregator 130.

At 210 patient state indications of the patient are semantically determined. Medical information is extracted from the state aggregator 130 and patient state indications that characterize a patient's medical and/or disease state are identified and normalized. The patient state indications can be obtained from the examination order entry or reason for the examination, and the patient problem list. In one embodiment, the patient state indications can include information about the examination. The extracted medical information can include structured or unstructured data. Patient state indications are identified by a semantic analysis of the extracted medical information. The semantic analysis normalizes the identified semantic concept according to one or more ontologies. Predetermined concepts according to one or more ontologies are identified as patient state indications. The identification can include a set matching, e.g. set intersection, or a rule based approach.

A relevancy score is computed and/or assigned for each lab value in one or more lab reports using mappings of the identified and normalized patient state indications and relevant lab values at 220. The mappings can include hierarchical reasoning using ontological concepts. The mappings are based on known relationships between patient state medical indications and relevant medical lab values, which are stored in the knowledge base 155. The computation/assignment of the relevancy score can include a rules based approach that determines the relevancy score. The computation can include a reconciliation of multiples relevancy scores from rules evaluation for a single lab value as a function of the multiple relevancy scores.

At 240 lab values can be displayed on the display device 180 according to the computed/assigned relevancy scores. The display can include lab values with a relevancy score greater than a predetermined threshold. The display can include lab values ordered or ranked according to relevancy. The display can include indications of the relevancy of each lab value, such as different coloring and/or intensities. In one embodiment, the lab values with relevancy scores are returned to another system for subsequent display and/or further manipulation.

The ordering and/or selection of individual acts are not intended to be limiting. The acts can be performed using the one or more configured processors 190. In some instances, the system and/or acts reduces the time to find and review lab values. In some instances, the system and/or acts reduces the time to review a medical image by refocusing attention to aspects of the medical image suggested by relevant lab values. In some instances, the relevant lab values can improve accuracy of a review of a medical image by confirming or refuting a potential diagnosis based on a combined review of the medical image and relevant lab values. In some instance, the relevant labs may suggest alternative diagnosis from a review of only a medical image.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system, comprising:
a relevancy computation engine configured to compute a relevancy score for a lab value in a lab report of a patient by applying rules that map one or more patient state indications and the lab value to the relevancy score.

2. The system according to claim 1, wherein the relevancy computation engine is further configured to compute relevancy scores for each lab value in the lab report; and further including:
a lab display configured to display the lab values on a display device according to the computed relevancy scores.

3. The system according to claim 2, wherein the lab display is further configured to filter the displayed lab values according to the computed relevancy scores and a predetermined threshold.

4. The system according to claim 1, further including:
a patient state extraction engine configured to identify and normalize the one or more patient state indications of the patient extracted from at least one of a reason for a medical examination and one or more patient medical problems.

5. The system according to claim 1, wherein the computed relevancy score includes a hierarchical reasoning of ontological concepts in mapping at least one of the one or more patient state indications.

6. The system according to claim 4, wherein the patient state extraction engine normalizes the one or more patient states using one or more medical ontologies.

7. The system according to claim 1, wherein the rules include an age of at least one patient state indication in computing the relevancy score.

8. The system according to claim 1, wherein the rules include an age of the lab value in computing the relevancy score.

9. The system according to claim 1, wherein the rules include placement of the lab value in a normal range for the lab value in computing the relevancy score.

10. The system according to claim 2, wherein the display of lab values includes at least one of: an ordering of displayed lab values according to the relevancy score, a ranking of displayed values according to the relevancy scores, or a highlighting of the displayed values according to the relevancy score.

11. A method, comprising:
computing a relevancy score for a lab value of a patient by applying rules that map one or more patient state indications and the lab value to the relevancy score.

12. The method according to claim 11, wherein computing includes computing a relevancy score for each lab value in a lab report; and further including:
displaying the lab values on a display device according to the computed relevancy scores.

13. The method according to claim 11, wherein displaying includes filtering the displayed lab values according to the computed relevancy scores and a predetermined threshold.

14. The method according to claim 11, further including:
identifying and normalizing the one or more patient state indications of the patient extracted from at least one of a reason for a medical examination and one or more patient medical problems.

15. The method according to claim 13, wherein semantic analysis includes hierarchical reasoning that generalizes extracted ontological concepts to identify and normalize the acute indications of the patient.

16. The method according to claim 11, wherein the computing includes hierarchical reasoning of ontological concepts in mapping at least one of the one or more patient state indications.

17. The method according to claim 11, wherein the rules include an age of at least one patient state indication in computing the relevancy score.

18. The method according to claim 14, wherein the rules include an age of the lab value in computing the relevancy score.

19. The method according to claim 12, wherein displaying includes at least one of: an ordering of displayed lab values according to the relevancy score, a ranking of displayed lab values according to the relevancy score, or a highlighting of the displayed lab values according to relevancy score.

20. A system, comprising:
a non-transitory storage media containing instructions that when executed by one or more processors are configured to:
identify and normalize one or more patient state indications of a patient using at least one of a reason for a medical examination and one or more patient medical problems;
compute relevancy scores for lab values of the patient by applying rules to the identified and normalized one or more patient state indications, wherein the rules map patient state medical indications and medical lab values to relevancy scores; and
display the lab values on a display device filtered by the relevancy score according to a predetermined threshold.
